(19) United States
(12) Patent Application Publication
(10) Pub. No.: US 2025/0009244 A1
(43) Pub. Date: Jan. 9, 2025

(54) DEVICES, SYSTEMS, AND METHODS FOR IMAGING LESIONS

(71) Applicant: BOSTON SCIENTIFIC SCIMED, INC., MAPLE GROVE, MN (US)

(72) Inventors: Jessica Bechly, Maple Grove, MN (US); Natalie White, Marlborough, MA (US); Alexander James Wiedmann, Columbia Heights, MN (US)

(73) Assignee: BOSTON SCIENTIFIC SCIMED, INC., MAPLE GROVE, MN (US)

(21) Appl. No.: 18/763,002

(22) Filed: Jul. 3, 2024

Related U.S. Application Data
(60) Provisional application No. 63/524,992, filed on Jul. 5, 2023.

(51) Int. Cl. A61B 5/055; A61B 5/0536; A61B 90/00

(52) U.S. Cl. CPC A61B 5/055; A61B 5/0536; A61B 90/39; A61B 2090/397; A61B 2090/3987

(57) ABSTRACT

An energy-delivering treatment system facilitating determination of various characteristics of a lesion and/or tissue at a treatment site, such as the size and/or location of the lesion; stiffness and/or elasticity of the tissue; impedance or resistance of the tissue (which may impact the form of energy such as electroporation and/or irreversible electroporation applied to the tissue); and/or the effects of a treatment applied to the lesion. Information gathered regarding the treatment site may be used to aid in predicting and measuring lesions, and/or in determining an appropriate treatment plan on a patient-by-patient basis. Markers may be used to facilitate locating of the treatment site after the energy-delivering assembly has been withdrawn from the treatment site.

### CROSS-REFERENCE TO RELATED APPLICATIONS

This application claims the benefit of priority under 35 U.S.C. § 119 to U.S. Provisional Application No. 63/524,992, filed Jul. 5, 2023, the entire disclosure of which is hereby incorporated by reference herein for all purposes.

### FIELD

The present disclosure relates generally to the field of devices, assemblies, systems, and methods utilizing an energy field to treat a patient. More particularly, the present disclosure relates to medical devices, assemblies, systems, and methods associated with therapies and treatments utilizing energy fields, including devices, assemblies, systems, and methods for imaging, sensing, locating, identifying, measuring, and determining treatment protocols utilizing energy fields.

### BACKGROUND

Various devices, assemblies, systems, and methods exist for energy-based medical treatment and/or therapeutic protocols. For instance, various focal therapy devices are configured to apply energy to debulk target tissue or to eliminate malignant cells. Various technologies for such therapy rely on thermal effects, such as radiofrequency (“RF”) heating, microwave heating, cryoablation, high intensity focused ultrasound (“HIFU”), etc. In contrast, electroporation and/or irreversible electroporation is a non-thermal therapy, and has significant potential benefits over thermal modalities. Energy may be applied to perform electroporation and/or irreversible electroporation (“IRE”) as a mode of treating various conditions and/or diseases using an energy field to interrupt and/or to change the nature of biological cellular matter. For instance, the applied electric field may significantly increase the electrical conductivity and permeability of the plasma in the cell membrane. The applied energy causes paths/pores to open within cell walls and/or membranes near the device applying the energy (e.g., near the electrode, probe, etc., thereof). In the case of reversible electroporation, pores in cell walls open to facilitate absorption of materials into the cells which may otherwise not readily pass through the cell walls and/or channels therethrough. The cells otherwise remain substantially intact. In contrast, in the case of irreversible electroporation, the electric field disrupts homeostasis and kills the cells, such as through apoptosis and/or necrosis. Various challenges with treatments involving use of energy fields include determining the appropriate parameters of energy to be applied to create the energy field, and the resulting effect on the cells thereafter. In particular, with IRE, the effect on the cells may not be detectable for several hours, and sometimes even as much 24-72 hours or more. Moreover, the cells which are affected may be a very small volume, and therefore difficult to locate and/or identify several hours after the treatment has already been performed. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an apparatus is configured to facilitate identification, characterization, and/or location of a site treated by application of therapeutic energy thereto. In accordance with various principles of the present disclosure, the apparatus, includes an energy-delivering assembly having an energy-delivering region along which an energy field is generated to apply therapeutic energy to a treatment site; and a device configured to characterize the treatment site for assessment by a medical professional in determining a treatment protocol generating an energy field with said energy-delivering region.

In some aspects, the device is configured to deploy a deployable marker at the treatment site. In some aspects, the marker is an injectable material delivered through a lumen delivered with said energy-delivering assembly. In some aspects, the energy-delivering assembly includes an energy-delivering member along which the energy-delivering region is defined, and the lumen is independent of said energy-delivering member. In some aspects, wherein the lumen is delivered to the treatment site with the energy-delivering member. Additionally or alternatively, the energy-delivering assembly includes an energy-delivering member defining the lumen through which the injectable is delivered therethrough. In some aspects, the marker is carried by the energy-delivering assembly. In some aspects, the marker is delivered to the treatment site as a part of the energy-delivering assembly and separated from the energy-delivering assembly to be deployed at the treatment site. In some aspects, the marker is carried over the energy-delivering assembly and slidable off the energy-delivering assembly to be deployed at the treatment site. In some aspects, the energy-delivering assembly defines a housing in which the marker is carried to the treatment site and from which the marker is released to be deployed at the treatment site.

In some aspects, device profiles a characteristic of the treatment site. In some aspects, the device uses one or more of elastography, magnetic resonance electrical impedance tomography, or hyperspectral imaging to map properties of the treatment site.

In accordance with various principles of the present disclosure, a system is configured to identify characteristics of a treatment site to aid in performing an energy-based treatment at the treatment site. In accordance with various principles of the present disclosure, the system includes an energy-delivering device comprising an energy-delivering assembly with an energy-delivering region along which an energy field is generated to apply therapeutic energy to a treatment site; a delivery device having a working channel through which the energy-delivering assembly is delivered to a treatment site; and a device configured to characterize the treatment site for assessment by a medical professional in determining a treatment protocol generating an energy field with the energy-delivering region.

In some aspects, device uses one or more of elastography, magnetic resonance electrical impedance tomography, or hyperspectral imaging to map properties of the treatment site, and said system further comprises a processor configured to receive information from said device and to develop models of the treatment site based on the received information.

In some aspects, device is configured to deploy a deployable marker at the treatment site. In some aspects, the deployable marker is identifiable after an energy field has been applied to the treatment site and the device has been removed from the treatment site.

In accordance with various principles of the present disclosure, a method for treating and characterizing a treatment site includes delivering an energy-delivering assembly of an energy-delivering device of an energy-delivering treatment system to a treatment site; generating an energy field along an energy-delivering region of the energy-delivering assembly to apply therapeutic energy to the treatment site; determining properties or characteristics of the treatment site with the energy-delivering treatment system; and using the determined properties to affect the treatment being performed, or further treatment to be performed with respect to the treatment site.

In some aspects, the method includes deploying a marker during or after application of therapeutic energy to the treatment site. wherein determining properties or characteristics comprises determining the location of the treatment site using the marker.

In some aspects, the method includes using the determined properties to create a three-dimensional map of the properties of the treatment site. In some aspects, the method includes using the map of the properties of the treatment site to aid prediction and measurement of therapeutic energy to be applied to a treatment site.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a schematic representation of an energy-delivering treatment system formed in accordance with aspects of the present disclosure in use with respect to a schematic representation of a treatment site.

FIG. 1A illustrates a schematic view along detail 1A in FIG. 1.

FIG. 2A illustrates an elevational view of an example of an embodiment of an energy-delivering assembly delivering an injectable marker.

FIG. 2B illustrates an elevational view of another example of an embodiment of an energy-delivering assembly delivering an injectable marker.

FIG. 3A illustrates an elevational view of an example of an embodiment of an energy-delivering assembly carrying a marker which is not injected into a treatment site.

FIG. 3B illustrates an elevational view of the energy-delivering assembly of FIG. 3A after deploying the marker with respect to a treatment site.

FIG. 4A illustrates an elevational view of an example of an embodiment of an energy-delivering assembly carrying a marker which is not injected into a treatment site.

FIG. 4B illustrates an elevational view of the energy-delivering assembly of FIG. 4A after deploying the marker with respect to a treatment site.

FIG. 5A illustrates an elevational view of an example of an embodiment of an energy-delivering assembly carrying a marker which is not injected into a treatment site.

FIG. 5B illustrates an elevational view of the energy-delivering assembly of FIG. 5A after deploying the marker with respect to a treatment site.

FIG. 6 illustrates, schematically, various techniques and methods which may be used with an energy-delivering treatment system in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure describes various improvements to medical procedures which apply energy to a treatment site within a patient. It will be appreciated that terms such as procedures, therapies, treatments, operations, protocols, etc., including other grammatical forms thereof, may be used interchangeably herein without intent to limit. It will further be appreciated that reference may be made interchangeably herein to a treatment site, a target site, an anatomical site, a therapy site, a lesion site, a tumor site, etc. (accompanied or not accompanied by the term “site”), without intent to limit. The following descriptions are generally with respect to energy treatment in the form of electroporation and/or irreversible electroporation (“IRE”), although applicable to other forms of energy treatments as well.

As generally used herein, the term “ablation” generally refers to removal of cells either directly or indirectly by supply of energy thereto, such as within an energy field such as an electric field, and may include removal by loss of cell function, cell lysis, coagulation, protein denaturation, necrosis, apoptosis, and/or irreversible electroporation. “Ablation” may similarly refer to creation of a lesion by ablation. Additionally, the terms “undesirable tissue,” “target cells,” “diseased tissue,” “diseased cells,” “tumor,” “cell mass” may be used herein to refer to cells removed or to be removed, in whole or in part, by ablation, and are not intended to limit application of any assemblies, systems, devices, or methods described herein. For example, such terms include ablation of both diseased cells and certain surrounding cells, despite no definite indication that such surrounding cells are diseased. Ablation performed by assemblies, systems, devices, or methods described herein may be of cells located around a biological lumen, such as a vascular, ductal, or tract area, for example, to create a margin for a medical professional to resect additional cells by ablation or other method. In accordance with various principles of the present disclosure, devices, assemblies, systems, and methods disclosed herein may be configured for performing ablation via electroporation and/or IRE.

Energy therapies such as electroporation and/or IRE typically involves applying an energizing potential to one or more electrodes positioned at a target site to create an electric field to which the target (e.g., undesirable tissue) is exposed. In the case of electroporation, the porosity of the cells at the target site is increased to allow absorption of materials, such as therapeutic materials, by the cells, with minimal effect on surrounding tissue. In the case of IRE, sufficient energy may be applied to destroy the cell wall, such as to destroy the lipid bilayer of the cell wall. In some aspects, energy is pulsed or otherwise varied to force the cell wall pores to open and close. After repeated opening and closing of the pores, and/or application of a certain amount energy, the cell walls may be permanently disrupted/damaged (e.g., broken), and internal components of and/or other substance from the cell may be released and enter into interstitial spaces and/or into the patient's body. Such components and/or substances (e.g., antigens) once released from the cells and within the body may then trigger the immune system to activate an immune response to treat the target site (e.g., tumor) further, such as by attacking the remaining undesirable tissue.

Energy for effecting a desired treatment may be applied to a target site via electrodes positioned adjacent, at, near, in, etc., the target site. The applied energy and/or energizing potential and/or the resulting energy field may be characterized by various parameters, such as, for example, frequency, amplitude, pulse width (duration of a pulse or pulse length), and/or polarity. Suitable energy sources include electrical waveform generators, such as waveform generators capable of creating IRE, high frequency IRE, NanoPulse, and/or ablative waveforms. The energy source generates an energy field with desired characteristics for the treatment to be performed at the target site, such as based on the treatment site, application, device, electrode configuration, etc. For instance, the energy field may be generated to have suitable characteristic waveform output in terms of voltage, impedance, frequency, amplitude, pulse width, delays between pulses, number of pulses per burst, number of bursts, and phase. Electric current may flow between the electrodes and through the tissue proportionally to the potential (e.g., voltage) applied to the electrodes. The supplied electric current provided by the energy source may deliver a pulse sequence to the target site. For example, an energy source may supply various waveforms in one or more pulse sequences tailored to the desired application

Electroporation and/or IRE therapy does not rely on thermal energy and may involve application of highly focused energy to a target site, therefore typically reducing and/or eliminating the risk of damage to surrounding cells which may be posed by other forms of energy treatments. Because of patient-to-patient variability, and tissue/tumor variability with a single patient, such as with regard to tissue properties (e.g., impedance, heterogeneity, the nature of a tumor to be treated, etc.), initial parameters for therapeutic energy protocols to be applied to a patient must be determined and assessed for each patient on an individual basis to achieve the desired effects of IRE. Accordingly, it is important to be able to determine and assess various parameters before applying energy protocols. The present disclosure describes various techniques for determining and assessing various tissue properties and other information and parameters used in determining a therapy to be performed. In accordance with various principles of the present disclosure, various tissue properties are imaged and mapped to aid in prediction and measurement of electroporation, IRE, and/or thermal lesions. Alternatively or additionally, information gathered in accordance with various principles of the present disclosure may then be further utilized, in accordance with various principles of the present disclosure, to inform computational models that display real time information such as estimations for thermal, IRE, and/or other lesion characteristics, such as sizes, such as for further treatment purposes.

The present disclosure describes further techniques for determining information about the treatment site, such as the lesion which has been created by the energy applied in accordance with various principles of the present disclosure. For instance, various techniques are disclosed in accordance with various principles of the present disclosure for determining and/or assessing information about the treatment site, such as, without limitation, the location of the lesion being treated and/or created; the size of the lesion; and/or the percentage of thermal damage vs. IRE at a given target site. In view of patient-to-patient variability, the applied treatment, even if properly assessed and delivered per techniques described herein prior to application to a patient, may nonetheless have different impact and/or effects from patient to patient. It is generally considered important if not crucial to characterize the treatment area after treatment has been performed (e.g., an IRE lesion which has been created) to be able to understand, assess, plan, etc., the care and/or further treatment to be provided to the patient.

Additionally, once electroporation and/or IRE has been performed, it generally is considered to be important to characterize the lesion which has been created to be able to understand the care to be provided to the patient. For instance, IRE typically spares the extracellular matrix of the cells to which energy has been applied. Even if the cells are dying, the matrix supporting the cells generally remains relatively intact. In accordance with various principles of the present disclosure, various devices, systems, and methods are disclosed for leaving a marker at a treatment site (e.g., within a tumor, such as within the extracellular matrix remaining intact even after IRE has been performed) that would remain in place after the energy treatment has been completed. The marker may facilitate later location and/or identification of the treatment site to assess the effect of the energy which had been applied to the treatment site. The marker may be a fluid (e.g., contrast agent, stain, dye, etc.) and/or an element with a generally fixed structure (in contrast with a fluid). The marker may be injected prior to, during, or after delivery of the therapeutic energy. The marker may remain in place at the treatment site at least a sufficient time to locate and/or identify the treatment site after the electrodes or other energy-delivery devices have been removed from the treatment site. For instance, the marker may remain in place for more than 24 hours, such as at least 72 hours, after the therapeutic treatment. In some aspects, the marker need not be actively removed by any methods other than natural bodily functions which may expel the marker and/or otherwise absorb the marker (e.g., in the case of a bioabsorbable marker).

Various embodiments of devices, assemblies, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

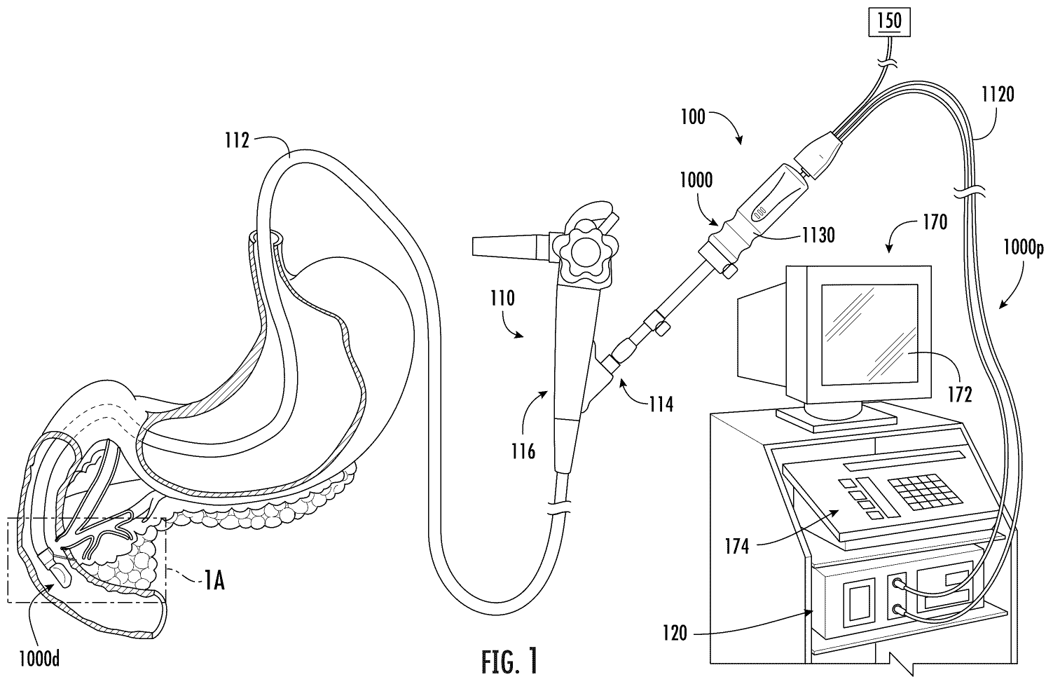

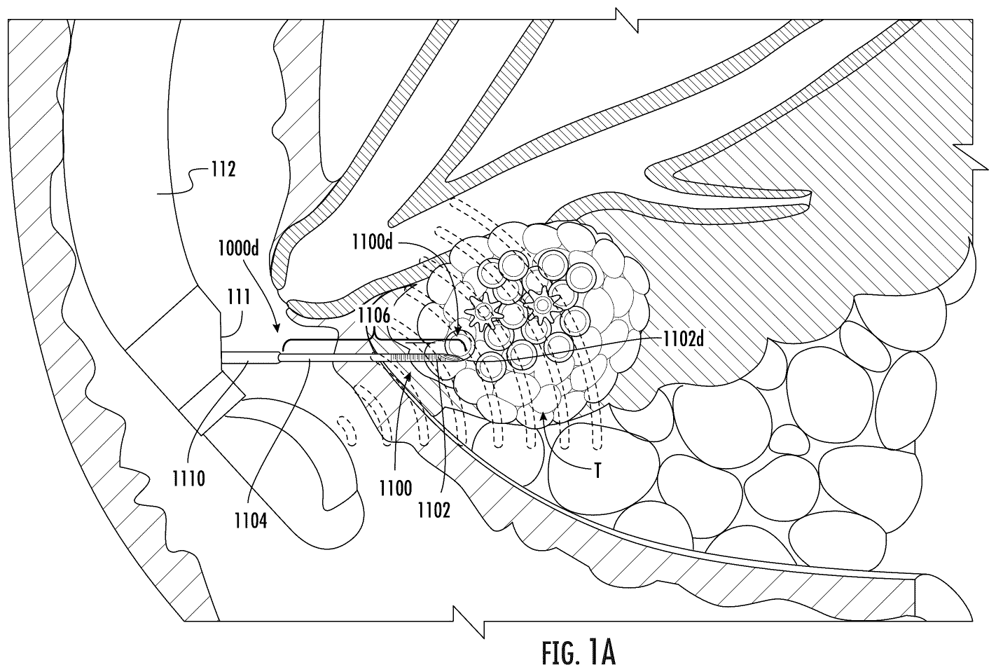

Turning now to the drawings, an example of an embodiment of an energy-delivering treatment system 100 configured to apply therapeutic energy to a treatment site within a patient is illustrated in FIG. 1. The energy-delivering treatment system 100 includes an energy-delivering device 1000, such as an electroporation device, configured to deliver therapeutic energy to a treatment site within a patient. In the example of an embodiment of an energy-delivering device 1000 illustrated in FIG. 1, an energy-delivering assembly 1100 (which may alternatively be referenced as a probe) is provided at a distal end 1000d of the energy-delivering device 1000, as illustrated in further detail in FIG. 1A. The energy-delivering assembly 1100 is deliverable within a patient's body (e.g., gastrointestinal system, as schematically illustrated in FIG. 1, or other anatomical site, the present disclosure not being limited in this regard) and is configured to establish a therapeutic energy field at a treatment site T.

As illustrated in FIG. 1A, showing a detail 1A of FIG. 1, the illustrated example of an embodiment of an energy-delivering assembly 1100 includes at least one energy-delivering member 1102 insertable with respect to a treatment site T (e.g., a tumor, lesion, etc.). The energy-delivering member 1102 may define an electrode therealong, such as along a distal end 1100d of the energy-delivering assembly 1100. As such, at least a portion of the energy-delivering member 1102 is formed of an electrically-conductive material such as medical grade stainless steel, platinum, gold, nitinol, a cobalt-chromium alloy, a nickel-cobalt alloy such as MP35N, or other alloys, or materials plated with electrically-conductive materials, etc. An insulation member 1104 may be provided proximal to the distal end 1102d of the energy-delivering member 1102 to define/delimit a distal energy-delivering region 1106 of the energy-delivering assembly 1100 along which an energy field is generated. In some aspects, the distal end 1100d (e.g., the terminal end) of the energy-delivering assembly 1100 has a sharp distal tip, such as formed at or along the terminal end of the energy-delivering member 1102. The sharp distal tip may be configured to pierce tissue/organs/tumor masses, such as in a manner known those of ordinary skill in the art. For instance, the energy-delivering member 1102 may be in the form of a trocar, a needle, or the like.

The energy-delivering assembly 1100 may be delivered to the treatment site T through a delivery device 110, such as schematically illustrated in FIG. 1. The delivery device 110 (e.g., tubular member, sheath, catheter, etc.) has a lumen or working channel therethrough sized to allow passage of the energy-delivering assembly 1100 therethrough. Additionally, the delivery device 110 is sized, shaped, configured, and/or dimensioned to be inserted into a human body, through a passage such as a natural anatomical passage, lumen, orifice, etc. (e.g., esophagus, stomach, intestines, etc., and/or a passage, lumen, channel, etc.) It will be appreciated that terms such as passage, lumen, orifice, channel, etc., may be used interchangeably herein without intent to limit. In the example of an embodiment of an energy-delivering treatment system 1000 illustrated in FIG. 1, the delivery device 110 is illustrated as an endoscope. However, the present disclosure need not be limited in this regard. As may be appreciated with reference to FIG. 1 and FIG. 1A, the delivery device 110 includes an insertion tube 112 defining a working channel 111 therethrough and through which the energy-delivering assembly 1100 is deliverable to the treatment site T. In the example of an embodiment illustrated in FIG. 1, the energy-delivering assembly 1100 of the energy-delivering treatment system 1000 is inserted into a port 114 of the delivery device 110, such as defined on a handle 116 of the delivery device 110, to access the working channel 111 (e.g., indicated in FIG. 1A) of the insertion tube 112.

The energy-delivering assembly 1100 may further include a sheath 1110 within/through which the energy-delivering assembly 1100 may be deliverable to the target site T, such as illustrated in FIG. 1A. The sheath 1110 may protect the passage through which the energy-delivering assembly 1100 is extended (e.g., the interior of the working channel 111 of the insertion tube 112 of the delivery device 110, and/or an anatomical passage or structure) from a sharp distal tip of the energy-delivering assembly 1100 (e.g., at the distal end 1102d of the energy-delivering member 1102). The sheath 1110 may be selectively proximally retractable with respect to the energy-delivering member 1102 and/or the energy-delivering member 1102 may be distally extendable with respect to the sheath 1110 to expose at least the distal energy-delivering region 1106 of the energy-delivering assembly 1100 to the treatment site T.

To apply therapeutic energy to the treatment site T, such as by generating an energy field (e.g., an electric field) along the energy-delivering region 1106 of the energy-delivering assembly 1110, the energy-delivering member 1102 is electrically coupled with an energy source 120. For instance, as illustrated in FIG. 1, a power connector 1120, extending along a proximal end 1000p of the energy-delivering device 1000, may electrically couple the energy-delivering member 1102 with an energy source 120 of the energy-delivering treatment system 100. The power connector 1120 may be wiring or other electrically-conductive member configured to be coupled to an energy source. The energy source 120 may be an electroporation generator (e.g., an electric field generator, a waveform generator, an electrical pulse generator, etc.) or any other energy-generating device such as known those of ordinary skill in the art for generating energy appropriate for application to an energy-delivering assembly 1100 of an energy-delivering device 1000 such for performing electroporation and/or IRE therapy. The present disclosure need not be limited by the details of the energy source.

In some aspects, the energy-delivering device 1000 further includes a handle 1130 from which the energy-delivering assembly 1100 extends distally, and from which the power connector 1120 extends proximally. The handle 1130 may be configured to control and/or adjust the position of the energy-delivering assembly 1100 and/or the sheath 1110, such as with respect to each other and/or with respect to the delivery device 110.

As discussed above, in accordance with various principles of the present disclosure, the example of an embodiment of an energy-delivering treatment system 100 illustrated in FIG. 1 is configured to facilitate identification, characterization, location, etc., of a treatment site T before, during, and/or after application of treatment energy thereto. For instance, in accordance with various principles of the present disclosure, the energy-delivering treatment system 100 may be configured to mark the treatment site T to facilitate identification, characterization, location, etc., thereof.

In some aspects, an energy-delivering treatment system 100 formed in accordance with various principles of the present disclosure is configured to inject a substance before (e.g., immediately before), during, and/or after (e.g., immediately after) delivering electroporation or IRE energy to a treatment site T. For instance, an injectable substance (e.g., fluid or otherwise) capable of being visualized or otherwise located and/or identified, such as by using visualizing and/or other equipment (e.g., an external visualization technique), may be injected into the treatment site T. Such injectable substance thereby allows for real-time and/or post-treatment identification of the treatment site T (e.g., an electroporated zone and/or thermal region). The injectable substance may be a contrast agent, stain, dye, cell marker, strain sensing mechanism, etc., which remains in the treatment site T after injection thereof. For instance, a cell which has succumbed to IRE may readily uptake such injectable substance. In some aspects, cells which die will turn one color, and those that live will turn another color.

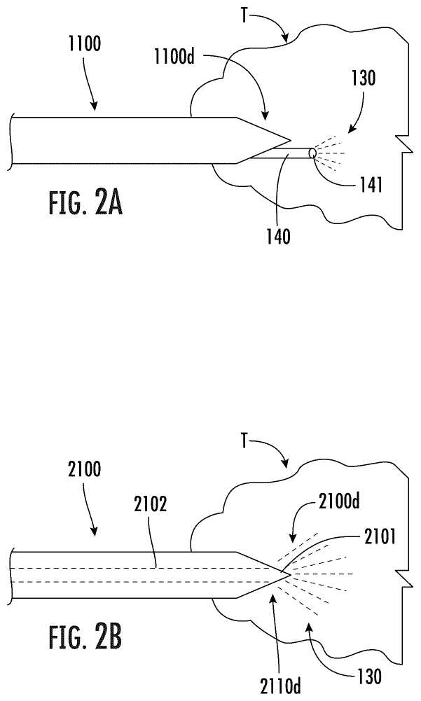

To deliver an injectable substance, an energy-delivering treatment system 100 formed in accordance with various principles of the present disclosure includes a delivery lumen through which an injectable substance may be delivered and deployed within a treatment site T. For instance, in some aspects, the energy-delivering device 1000 may include a delivery lumen. In the example of an embodiment of an energy-delivering assembly 1100 illustrated in FIG. 2A, an injectable substance 130 (which optionally may be considered a part of an energy-delivering treatment system 100 formed in accordance with various principles of the present disclosure) may be injected via a lumen formed separately from the energy-delivering member 1102 and even separately from the energy-delivering assembly 1100. For instance, as illustrated in FIG. 2A, a separate tubular member 140 may be inserted with the energy-delivering assembly 1100 to the treatment site T. For instance, the separate tubular member 140 may be inserted through the working channel 111 of a delivery device 110 (e.g., an endoscope 110) such as illustrated in FIG. 1 and FIG. 1A. Alternatively, a device exchange could be performed, and a tubular member, such as a separate hollow needle (e.g., not used for or during ablation), can be passed down the working channel 111 of the delivery device 110. The injectable substance 130 of the energy-delivering treatment system 100 may be delivered to the tubular member in a manner as known to those of ordinary skill in the art, such as via the port 114 of the delivery device 110.

Additionally or alternatively, the energy-delivering device 1000 may have an energy-delivering assembly with a delivery lumen defined through a component thereof for delivering an injectable substance 130 to a treatment site T. For instance, a lumen may be incorporated into the energy-delivering device 1000 illustrated in FIG. 1 and FIG. 1A. More particularly, a lumen 2101 may be defined through the energy-delivering member 2102 of an energy-delivering assembly 2100 such as illustrated in FIG. 2B. It will be appreciated that the example of an embodiment of an energy-delivering assembly 2100 illustrated in FIG. 2 may extend from a distal end 1000d (e.g., form a part of or otherwise be incorporated into) the example of an embodiment of an energy-delivering device 1000 of the example of an embodiment of an energy-delivering treatment system 100 illustrated in FIG. 1. Once the distal end 2100d of the energy-delivering assembly 2100 has entered the treatment site T, an injectable substance 130 may be delivered to the treatment site T via the lumen 2101, such as illustrated in FIG. 2. In accordance with various principles of the present disclosure, the injectable substance 130 may be injected into the treatment site T before, during, or after application of treatment energy to the energy-delivering assembly 2100 to create an energy field configured to treat the treatment site T. Optionally, the injectable substance 130 is delivered to the lumen 2101 via the handle 1130 from a fluid source 150 fluidly coupled with the handle 1130, such as in a manner known to those of ordinary skill in the art.

Like the above-described energy-delivering assembly 1100 illustrated in FIG. 1A, the example of an embodiment of an energy-delivering assembly 2100 illustrated in FIG. 2 may have a sharp distal end 2100d configured to enter into the treatment site T (e.g., to enter a tumor). It will be appreciated that various other features and/or structures of the example of an embodiment of an energy-delivering assembly 2100 illustrated in FIG. 2 may be similar or substantially the same as features and/or structures of the example of an embodiment of an energy-delivering assembly 1100 illustrated in FIG. 1A. For the sake of brevity, and without intent to limit, reference is made to the above-description of such additional features and structures as applicable to the energy-delivering assembly 2100 illustrated in FIG. 2.

In accordance with various principles of the present disclosure, the injectable substance 130 may be any of a variety of substances allowing detection (visualization) thereof in any of a variety of manners. In some aspects, the injectable substance 130 may include a contrast agent allowing visualization thereof and of the treatment site T with computed tomography (CT). Such embodiment would enable real-time CT imaging and real-time volumetric lesion assessment. In accordance with various principles of the present disclosure, the injectable substance 130 is injected into the treatment site T and imaged immediately after or during treatment delivery. In accordance with various principles of the present disclosure, care should be taken to assure that the contrasted/dyed/stained areas align with the ablated areas, or various levels of applied electroporation.

Additionally or alternatively, the injectable substance 130 may include a fluorescent stain allowing visualization thereof and of the treatment site T using lasers. For instance, the injectable substance 130 may be a fluid made of and/or containing molecules that fluoresce at different selected light wavelengths. Such molecules would be designed to be taken up by cells at the treatment site T. One example of such molecule is propidium iodine, which could be taken up by cells that have been electroporated, and which will visually highlight the IRE ablation area. To visualize the treatment site T endoscopically, lasers may be used to emit light within the tumor and to look for fluorescent molecules. A similar process may be used in an open procedure.

Additionally or alternatively, the injectable substance 130 may include a contrast agent and/or fluorescent stain visualizable with photoacoustic imaging. During photoacoustic imaging, laser pulses are delivered into the tissue. Some of the laser energy is converted into heat, which expands the tissue, allowing light waves and pressure waves to facilitate visualization. In accordance with various principles of the present disclosure, some media (possibly contrast or a fluorescent stain) could be used to preferentially visualize either the IRE or thermal lesion. Care should be taken to correlate stained areas with tissue changes (levels of electroporation, thermal ablation, etc.).

Additionally or alternatively, the injectable substance 130 may include electric field responsive dyes/contrasts visualizable using fluoroscopy and/or endoscopic ultrasound. Such injectable substance 130 may or may not be not taken up by cells. Instead, the molecules themselves change in response to an energy field, such as an electric field applied during IRE treatment. As energy treatment is applied to a treatment site T in which has been deposited an injectable substance 130 in the form of electric field responsive dyes/contrasts, the molecules of the injectable substance 130 would resonate and emit light when exposed to a laser of a certain wavelength or a signal of a certain voltage. In some aspects, dyes with different thresholds for emission may be combined to create a more nuanced image. In some aspects, the dyes are visible, and may be selected so that their emission/emissions correlate with the IRE lesion area.

In some aspects, the injectable substance 130 may include a thermochromic dye visualizable using an endoscope, such as during application of a thermal energy treatment. The thermochromic dyes may be selected to change color when subjected to certain temperatures. Injection of such dyes into the treatment site T before application of treatment energy, and visualization of such dyes (e.g., with an endoscope) will provide a visual, chromatic map of any thermal tissue damage.

In addition to or instead of injecting an injectable substance, such as described above, one or more markers may be deployed in a manner other than by injection to facilitate identification, location, etc., of a treatment site. Thus, in contrast with an injectable substance which may be generally fluid and may not have a fixed shape (e.g., may vary in shape, such as by taking on the shape of a vessel in which it is contained and/or a lumen through which it is injected), an additional or alternative marker having a generally non-fluid form (e.g., a gel, a biocompatible film, a series of nanobubbles, etc.) may be deployed by an energy-delivering treatment system 100 formed in accordance with various principles of the present disclosure. For instance, an additional or alternative marker which is not intended to be injected may be carried on a portion of the energy-delivering assembly of the energy-delivering treatment system 100, such as on an exterior portion of the energy-delivering assembly. Such marker may be formed of a visualizable material such as a radiopaque material (visible under x-ray), or a material otherwise configured to be compatible with various imaging modalities. In some aspects, the marker is deployed after ablation for site identification of the treated area of the treatment site T. In some aspects, the marker is a biodegradable marker, such as not to leave a long-term foreign body in the patient. The marker may be formed of a polymer, metal, or other visualizable (e.g., radiopaque material) such as known those of ordinary skill in the art. In some aspects, the marker may not facilitate visualization of the entire area of the treatment zone, but, instead, would aid in more specified site identification of the ablated tissue at the treatment site (e.g., location of dead cells). Such a deployable marker configured to be deployed other than by injection in accordance with various principles of the present disclosure may have any of a variety of possible configurations. For the sake of convenience, and without intent to limit, such marker is referenced herein as a non-fluid marker, to be deployed with respect to a treatment site in a manner other than by injection, in contrast with the above-described injectable markers. Additionally or alternatively, such marker may be referenced herein as a deployable marker carried by an energy-delivering assembly, since the energy-delivering assembly is generally the component or member of and energy-delivering treatment system formed in accordance with various principles of the present disclosure which is delivered to a treatment site. However, it will be appreciated that reference to a non-fluid marker being carried by an energy-delivering assembly is not necessarily limited to non-fluid forms and/or to being carried only by an energy-delivering assembly. It will be appreciated that the following examples of embodiments are only some of a variety of forms of a deployable marker, and the marker may be any a variety of other shapes (cylindrical, spherical, prismatic, etc.), made of a variety of other materials, and/or delivered in a variety of other manners (via active or passive mechanisms), the principles of the present disclosure not being limited by the examples of embodiments described herein.

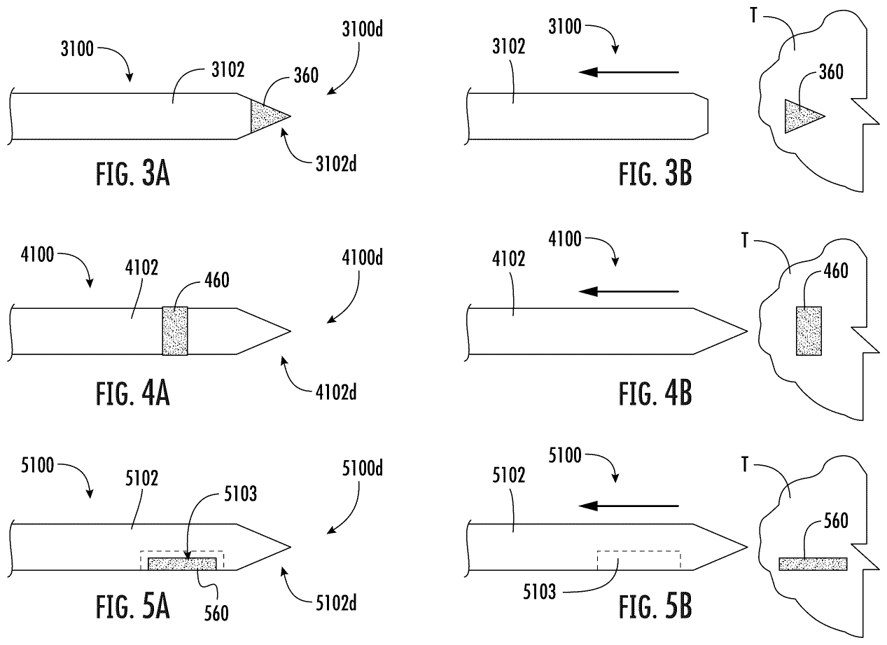

An example of an embodiment of an energy-delivering assembly 3100 configured to carry, deliver, and deploy a non-fluid deployable marker 360 at a treatment site T is illustrated in FIG. 3A. It will be appreciated that the illustrated example of an embodiment of an energy-delivering assembly 3100 may extend from a distal end 100d of an energy-delivering treatment system 100 such as illustrated in FIG. 1. For the sake of brevity, reference is made to the description of the energy-delivering treatment system 100 illustrated in FIG. 1 as an example of an embodiment of an energy-delivering treatment system of which the example of an embodiment of an energy-delivering assembly 3100 illustrated in FIG. 3A is a component. The example of an embodiment of an energy-delivering assembly 3100 illustrated in FIG. 3A carries a non-fluid deployable marker 360 formed in accordance with various principles of the present disclosure. The non-fluid deployable marker 360 illustrated in FIG. 3A is separable and deployable from the energy-delivering assembly 3100. For instance, the non-fluid deployable marker 360 may form or otherwise be coupled to the distal end 3100d of the energy-delivering assembly 3100. The non-fluid deployable marker 360 is configured to be separable from the energy-delivering assembly 3100 in any of a variety of manners. For instance, the non-fluid deployable marker 360 may be a break-away tip of the energy-delivering assembly 3100. For example, in some embodiments, a mechanical actuator, such as a button, may extend from the non-fluid deployable marker 360 to a location external to the patient's body (e.g., on the handle 116 of the delivery device 110) and may be movable to push the deployable marker 360 out of its place with respect to the energy-delivering assembly 3100. In other embodiments, the energy-delivering assembly 3100 may be manipulated (e.g., rotated, bent, torqued, etc.) to apply stresses to the non-fluid deployable marker 360 to cause an intentional breakage. In some embodiments, the non-fluid deployable marker 360 may be supported during delivery, but proximal retraction/withdrawal of the energy-delivering member 3102 may cause removal of a support structure, thereby allowing the non-fluid deployable marker 360 to fall or break away from the retracted portion of the energy-delivering member 3102. Alternatively or additionally, proximal retraction/withdrawal of the energy-delivering member 3102 may cause the non-fluid deployable marker 360 to snag or catch on another component, allowing the non-fluid deployable marker 360 to fall or break away from the retracted portion of the energy-delivering member 3102. Additionally or alternatively, the energy-delivering member 3102 of the energy-delivering assembly 3100 may define a lumen therethrough, with a proximal end of the non-fluid deployable marker 360 fitted therein (e.g., by a break-away connection, a friction fit, a snap fit, etc.). A pusher, such as a stylet or other elongated member known those of ordinary skill in the art, may be translatable through the lumen of the energy-delivering member 3102 to distally push the proximal end of the non-fluid deployable marker 360 out of the lumen to deploy the non-fluid deployable marker 360 with respect to the treatment site T. Once the non-fluid deployable marker 360 has been separated from the energy-delivering assembly 3100 and deployed at the treatment site T, the energy-delivering assembly 3100 may be retracted proximally and removed from the treatment site T, such as illustrated in FIG. 3B.

An example of an embodiment of an energy-delivering assembly 4100 configured to carry an additional or alternative non-fluid deployable marker 460 to deliver and deploy such non-fluid deployable marker 460 with respect to a treatment site T is illustrated in FIG. 4A. It will be appreciated that the illustrated example of an embodiment of an energy-delivering assembly 4100 may extend from a distal end 100d of an energy-delivering treatment system 100 such as illustrated in FIG. 1. For the sake of brevity, reference is made to the description of the energy-delivering treatment system 100 illustrated in FIG. 1 as an example of an embodiment of an energy-delivering treatment system of which the example of an embodiment of an energy-delivering assembly 4100 illustrated in FIG. 4A is a component. The example of an embodiment of an energy-delivering assembly 4100 illustrated in FIG. 4A is configured to carry a non-fluid deployable marker 460 movable with respect thereto. For instance, the non-fluid deployable marker 460 may be movably or removably (e.g., slidably or otherwise) mounted with respect to (e.g., on top of, over such as circumferentially over or around, etc.) a portion of the energy-delivering assembly 4100. For instance, in the example of an embodiment illustrated in FIG. 4A, the non-fluid deployable marker 460 is illustrated as slidably mounted on the energy-delivering member 4102 of the energy-delivering assembly 4100. In some aspects, the non-fluid deployable marker 460 may be mounted proximally spaced from the distal end 4100d (e.g., the free, terminal end) of the energy-delivering assembly 4100. The non-fluid deployable marker 460 is configured to be movable with respect to the energy-delivering assembly 4100 to be deployed therefrom in any of a variety of manners. For instance, the non-fluid deployable marker 460 may be in the form of a band or ring extending generally circumferentially around a portion of the energy-delivering assembly 4100 delivered to a treatment site T. A pusher, such as a stylet or other appropriate member known those of ordinary skill in the art, may be translatable with respect to (e.g., alongside) the energy-delivering assembly 4100 to distally push the non-fluid deployable marker 460 with respect to (and, generally off of) the energy-delivering assembly 4100 to deploy the non-fluid deployable marker 460 with respect to the treatment site T. The pusher optionally is delivered through a delivery channel of a delivery device through which the energy-delivering assembly 4100 is delivered to the treatment site T, and/or associated with (e.g., movably coupled with respect to) the energy-delivering assembly 4100. In some aspects, the pusher may be delivered alongside/outside the energy-delivering assembly 4100, acting like a moveable sheath that pushes the non-fluid deployable marker 460 off the energy-delivering member 4102. Once the non-fluid deployable marker 460 has been separated from the energy-delivering assembly 4100 and deployed at the treatment site T, the energy-delivering assembly 4100 may be retracted proximally and removed from the treatment site T, such as illustrated in FIG. 4B.

Yet another example of an embodiment of an energy-delivering assembly 5100 configured to carry an additional or alternative non-fluid deployable marker 560 to deliver and deploy such non-fluid deployable marker 560 at a treatment site T is illustrated in FIG. 5A. It will be appreciated that the illustrated example of an embodiment of an energy-delivering assembly 5100 may extend from a distal end 100d of an energy-delivering treatment system 100 such as illustrated in FIG. 1. For the sake of brevity, reference is made to the description of the energy-delivering treatment system 100 illustrated in FIG. 1 as an example of an embodiment of an energy-delivering treatment system of which the example of an embodiment of an energy-delivering assembly 5100 illustrated in FIG. 5A is a component. The example of an embodiment of an energy-delivering assembly 5100 illustrated in FIG. 5A is configured to carry a non-fluid deployable marker 560 movable with respect thereto. For instance, the non-fluid deployable marker 560 may be mounted with respect to a portion of the energy-delivering assembly 5100, such as in a housing 5103 or other structure formed with respect to the energy-delivering assembly 5100 and configured to carry the non-fluid deployable marker 560. For instance, in the example of an embodiment illustrated in FIG. 5A, the housing 5103 is a recess formed on the exterior of a portion or component of the energy-delivering assembly 5100, such as along an exterior of an energy-delivering member 5102 of the energy-delivering assembly 5100. The non-fluid deployable marker 560 is movably/removably mounted with respect to the energy-delivering assembly 5100 to be separated therefrom to be deployed with respect to a treatment site T, such as illustrated in FIG. 5B. A pusher, such as a stylet or other appropriate member known those of ordinary skill in the art, may be movable with respect to the energy-delivering assembly 5100 to move the non-fluid deployable marker 560 with respect to (and, generally off of) the energy-delivering assembly 5100 to deploy the non-fluid deployable marker 560 with respect to the treatment site T. The pusher may be coupled to an actuator, such as a button, on a handle of the energy-delivering assembly 5100. In some embodiments, a spring is provided which may be compressible to apply force to the non-fluid deployable marker 560 to deploys/ejects the non-fluid deployable marker 560 from the energy-delivering assembly 5100. The pusher optionally is delivered through a delivery channel of a delivery device through which the energy-delivering assembly 5100 is delivered to the treatment site T, and/or associated with (e.g., movably coupled with respect to) the energy-delivering assembly 5100. Once the non-fluid deployable marker 560 has been separated from the energy-delivering assembly 5100 and deployed at the treatment site T, the energy-delivering assembly 5100 may be retracted proximally and removed from the treatment site T, such as illustrated in FIG. 5B.

As noted above, in accordance with various principles of the present disclosure, one or more markers such as described above may be deployed before, during, and/or after treatment of a treatment site in accordance with various principles of the present disclosure. The marker facilitates location of the treatment site after the energy-delivering assembly used to apply the treatment energy has been removed. Because the treatment site may be much smaller than the devices or elements used to apply therapeutic energy thereto, markers such as those disclosed herein may be useful in facilitating identification of the location of a treatment site to evaluate the effect of the treatment on the treatment site, such as to determine further treatment, therapies, protocols, etc., to be performed.

It will be appreciated that to determine the initial treatment to be performed at a treatment site and/or to determine further treatment, therapy, protocols, etc., to be performed, medical professionals may use various forms of information regarding properties, characteristics, etc., of the treatment site. In accordance with various principles of the present disclosure, an energy-delivering treatment system 100 formed in accordance with various principles of the present disclosure, and illustrated schematically in FIG. 1, includes a processor 170 configured to receive information from the treatment site and/or to present such information to a medical professional and/or to control or otherwise affect the therapeutic treatment to be applied to a treatment site. In some aspects, the processor 170 may control fluid injection into the target site if fluid source/reservoir is operatively coupled with the processor 170. In the example of an embodiment illustrated in FIG. 1, the processor 170 includes a console 172 configured to display information for use by a medical professional. The console 172 and/or an input device such as a keyboard 174 may be coupled with the processor 170 and configured to allow input of information to the processor 170, such as by the medical professional. Additionally, information with regard to the treatment site is provided to the processor 170 via the energy-delivering device 1000 and or further instruments or devices of the energy-delivering treatment system 100 such as known those of ordinary skill in the art.

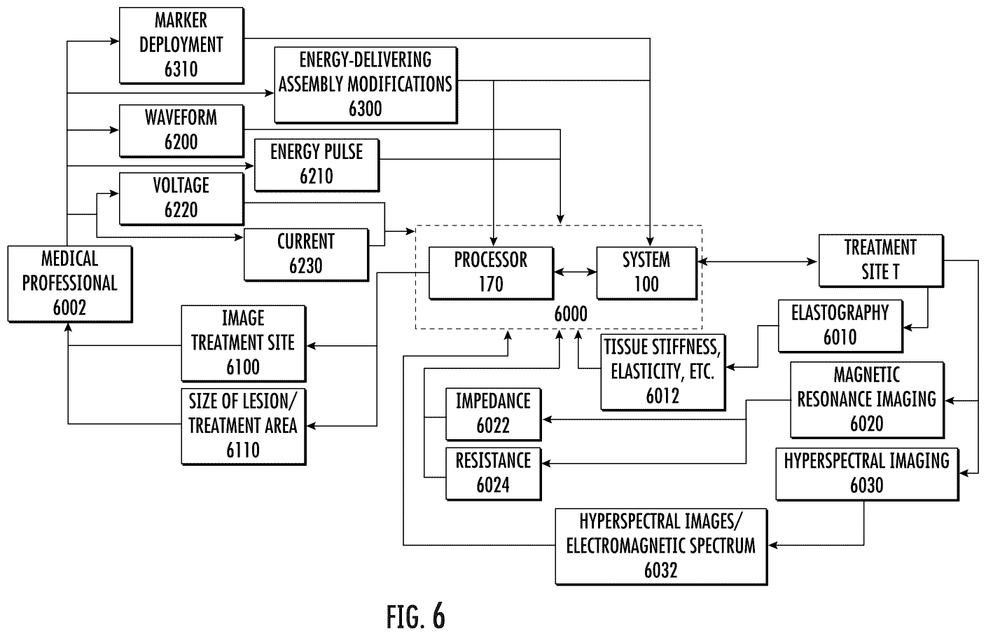

In accordance with various principles of the present disclosure, various techniques may be used to obtain information about a treatment site, such as for evaluating, assessing, determining one or more properties, etc., of the treatment site; detecting and/or otherwise determining the location and/or mapping (including, without limitation, size, area, volume, and/or shape) of the treatment site and/or of a diseased area (e.g., lesion) to be treated; obtaining information (e.g., diagnostic) about the nature or condition of the treatment site; assessing the disease state of the treatment site; determining appropriate treatments to apply; predicting effects of treatments to apply; evaluating, measuring, tracking, etc., the effects of treatments performed with respect to the treatment site; and other relevant uses with regard to treatments using energy fields. In some aspects, various techniques may be used to create a map, such as a three-dimensional map, of a treatment site. Assessments of the treatment site may be to determine the nature, characteristics, parameters, etc., of the therapeutic energy treatment to be applied and/or to assess the effects of such treatment after application thereof to a treatment site. Various techniques which may be used in accordance with various principles of the present disclosure to gather information with regard to the treatment site and/or to affect/control the therapy to be applied to the treatment site T are illustrated schematically in FIG. 6. Such information may be obtained with one or more elements, members, devices, assemblies, etc., of an energy-delivering treatment system 100 formed in accordance with various principles of the present disclosure, such as illustrated schematically in FIG. 1 and in FIG. 6 by block 6000, and described in further detail below. The information obtained from the treatment site T may be transferred to a processor 170 of the energy-delivering treatment system 100, such as further illustrated schematically in FIG. 1 and in FIG. 6. The information may be made available to a medical professional, as illustrated schematically by block 6002. The medical professional may use the information which has been gathered to control or otherwise to affect the therapy protocol to be carried out, such via input into the processor 170 (such as via the keyboard 174 schematically illustrated in FIG. 1). The processor 170 may then process and control one or more elements, members, devices, assemblies, etc., of the energy-delivering treatment system 100 to effect the desired therapy, such as in accordance with the input provided by the medical professional. Although the present disclosure generally refers to therapeutic energy treatments in the form of electroporation and/or IRE, other forms of treatments are within the scope and spirit of the present disclosure. Moreover, although the present disclosure refers to lesions caused by electroporation and/or IRE, the lesions may additionally or alternatively be other types of lesions, such as thermal lesions. Various techniques for gathering information about a treatment site T and/or carrying out a protocol (which may be determined based on the gathered information) are described with reference to the schematic illustrations in FIG. 6.

In accordance with various principles of the present disclosure, as illustrated schematically in FIG. 6, elastography 6010 (including, but not limited to, shear wave elastography) may be used to gather information about a treatment site T. For instance, elastography 6010 may be used to profile tissue stiffnesses such as by deforming tissue and observing the tissue response in order to assess tissue stiffness and/or elasticity and/or, in some instances, density. In accordance with various principles of the present disclosure, tissue stiffness, etc., determined by elastography is correlated with evidence of IRE and/or thermal damage, or other effects of therapeutic treatment, such as to measure lesion size after therapeutic treatment thereof. Deformation of the tissue to determine properties thereof, such as stiffness, may be performed in a variety of manners, such as, without limitation, via physical probing, acoustic imaging, during observation of one or more bodily functions or processes, etc. In some aspects, physical probing may be performed by pushing the tissue, and/or moving the tissue around with a component of an energy-delivering treatment system formed in accordance with various principles of the present disclosure, such as with a component of an energy-delivering device formed in accordance with various principles of the present disclosure. In some aspects, acoustic imaging may be performed with the use of an ultrasound-emitting probe which creates pressure waves which impact the tissue to deform the tissue. An element impacting the tissue to deform the tissue (e.g., a physical probe or acoustic probe) may be delivered to the treatment site transluminally, and optionally with an energy-delivering device formed in accordance with various principles of the present disclosure. Additionally or alternatively, deformation of tissue and/or differentiation of a region of interest within the patient may be as a result of movements in the patient's body and/or observation of movements in the patient's body, such as movements due to physiological processes (e.g., peristalsis along the gastrointestinal tract, beating of the heart, etc.). Additionally or alternatively, various properties of the tissue, such as tissue stiffness and/or elasticity, may be measured using pressure/stress sensors (e.g., delivered transluminally, endoscopically, etc.), ultrasound, and/or magnetic resonance imaging (MRI). Information regarding tissue stiffness, elasticity, etc., such as determined by elastography, is indicated schematically by block 6012, as being transmitted to the processor 170 at block 6000 and/or provided to the medical professional at block 6002. In some aspects, the processor 170 is configured for data processing, such as for imaging modalities (e.g., MRI, elastography, hyperspectral imaging, etc.). The processor of a tubular elongate member in the form of an endoscope may be operatively associated or may remain independent of the processor 170 of the system as a whole. The transmitted information may be used to determine and/or understand the tissue properties; to predict lesion sizes and/or areas (such as prior to treatment); and/or to visualize and/or assess lesion sizes after treatment (e.g., with the assistance of dyes, aids, etc., such as described above). As may be appreciated, any of the above-described markers may be used in conjunction with such profiling, assessment, etc.

In some aspects, further information with regard to a treatment site useful in developing a treatment protocol may be obtained via magnetic resonance electrical impedance tomography 6020 (MR EIT). For instance, MR EIT may be used to create a three-dimensional impedance map of a treatment site (e.g., tissue at the treatment site). As may be appreciated by those of ordinary skill in the art, lesion size is typically highly dependent on tissue impedance. Moreover, lower tissue impedance generally indicates an IRE-affected zone. Thus, in accordance with various principles of the present disclosure, mapping of three-dimensional tissue impedance may be used to enable prediction of IRE and thermal lesion zones prior to application of therapeutic energy to a treatment site. Additionally or alternatively, mapping of three-dimensional tissue impedance may be used to assess the effect of a therapeutic treatment, such as to assess an IRE lesion size post treatment. In accordance with various principles of the present disclosure, an energy-delivering treatment system, such as an energy-delivering device, formed in accordance with various principles of the present disclosure may include an MRI-compatible endoscopic probe to create such map. The probe may be inserted with, as a part of or as separate component from, the energy-delivering assembly used to apply the therapeutic energy field if the impedance map is to be created prior to or immediately after treatment. Impedance measurements 6022 and/or resistance measurements 6024 obtained in this manner may transmitted to the processor 170 at block 6000 and/or provided to the medical professional at block 6002. In some aspects, a medical professional may or may not have to choose each specific parameter or aspect of the waveform. For example, the medical professional may choose from a selection of pre-determined waveforms (instead of setting the waveform, voltage, current, etc.). The final variable may be calculated by the processor 170 so that the desired value of the chosen variable is generated.

In accordance with various further principles of the present disclosure, hyperspectral imaging 6030 may be used, in addition or instead of the above techniques, to gather data with respect to properties of the treatment site. For instance, hyperspectral imaging may be used to visualize the electromagnetic spectrum of the tissue with very fine wavelength resolution. In accordance with various principles of the present disclosure, changes in the electromagnetic spectrum could be correlated to differences in tissue properties and features-possibly even thermal damage and/or IRE-affected zones. In accordance with various principles of the present disclosure, an energy-delivering treatment system, such as an energy-delivering device, formed in accordance with various principles of the present disclosure may include an imaging tip capable of hyperspectral imaging of a treatment site. The imaging tip may be inserted separately from or with (e.g., as a part of or as separate component from) the energy-delivering assembly used to apply the therapeutic energy field to the treatment site. Hyperspectral images of the electromagnetic spectrum 6032 obtained in this manner may be used by the processor 170 at block 6000 and/or provided to the medical professional at block 6002

Using any of the above-described techniques, and/or other techniques, after a treatment site has been assessed, information about the treatment site T (e.g., the nature and characteristics and/or other properties of the treatment site T) may be processed, such as by a processor 170 at block 6000, and/or provided to a medical professional at block 6002 of FIG. 6. For example, as illustrated schematically in FIG. 6, an image of the treatment site (represented by block 6100), a computation (including a prediction, such as pre-treatment, and/or a visualization, such as post-treatment) of the size of the lesion/treatment area (represented by block 6110), or other information impacting treatment protocol decisions, may be delivered to and/or displayed for the medical professional. The medical professional may use such information to determine a desired treatment protocol. For instance, to achieve the desired treatment of a treatment site T, various parameters of energy to be delivered to generate a therapeutic energy field at the treatment site T must be assessed, determined, selected, and conveyed to the energy-delivering treatment system 100 to carry out the desired protocol. For instance, the medical professional typically must choose the appropriate energy waveform 6200, energy pulse characteristics 6210, voltage 6220 or current 6230 (it will be appreciated that medical professionals typically can adjust either the voltage or the current of the applied energy, the resistance being set by the patient, such as by the properties of their tissue), etc., which is input into the processor 170 and/or directly into the energy-delivering treatment system 100 represented by block 6000, to create an energy field capable of achieving the desired therapeutic effect at the treatment site T. Additionally or alternatively, the medical professional may use the information obtained in accordance with various principles of the present disclosure to make various adjustments to the energy-delivering assembly (represented by block 6300), such as via the energy-delivering treatment system 100 and/or directly via the processor 170. Additionally or alternatively, the medical professional may use the information obtained in accordance with various principles of the present disclosure to deploy one or more markers such as one or more of the markers 130, 360, 460, 560 described herein, as represented by block 6310.

In accordance with various principles of the present disclosure, various information gathered with an energy-delivering treatment system 100, such as described herein or otherwise, may be used to generate three-dimensional tissue mapping, such as from any of the above-described techniques, to inform and/or be used in compiling and creating computational models that display real-time estimations for the effect of a proposed treatment protocol on a treatment site, such as, without limitation, predicting thermal and/or IRE lesion sizes. A computational model may display (e.g., via a console 172 or other information display medium, such as a print-out) a prediction based on the selected energy output to be delivered to a treatment site T in view of the nature, properties, characteristics, etc. of the treatment site T. For instance, a computational model may display a prediction based on the measured tissue properties and the chosen waveform values to be delivered to the treatment site. A medical professional may thereby be enabled to visualize and to at least roughly estimate a treatment plan before actually proceeding with such treatment plan. It will be appreciated that the above principles of the present disclosure allow medical professionals to choose waveform and other energy input values on a patient-to-patient basis. Such customizability for individual patients afforded by the present disclosure is particularly advantageous in view of patient-to-patient heterogeneity and other variances which may dramatically influence both IRE as well as thermal lesion sizes. The ability to locate and to estimate the size of both IRE as well as thermal lesions in accordance with various principles of the present disclosure also enables medical professionals to avoid damaging key/critical anatomical structures (e.g., blood vessels) that may be close to (e.g., potentially within the range of the energy field to be applied to) the treatment site of interest.

It will be appreciated that the energy-delivering assembly used to deliver energy-based treatment in accordance with various principles of the present disclosure may be any appropriate form or configuration for delivering energy-based therapy. In some aspects, the energy-delivering assembly may be a bipolar probe, such as a linear bipolar probe with both electrodes delivered to the treatment site axially spaced apart from each other on the same energy-delivering assembly. However, it will also be appreciated that configurations other than bipolar and/or other than linear are within the scope and spirit of the present disclosure as well. For instance, energy-delivering assemblies with more than two electrodes, or unipolar and/or monopolar devices may be used with energy-delivering treatment systems formed in accordance with various principles of the present disclosure. And, principles of the present disclosure may be applied to energy other than electroporation and/or IRE energy, such as other energy sources for ablation or otherwise.

It will further be appreciated that any or all of the aspects of the above-described delivery and deployment may be performed using available appropriate imaging techniques such as known to those of ordinary skill in the art. Additionally, it will be appreciated that the present disclosure is not limited by the type of processor, monitor, delivery device, or other devices, systems, equipment, etc., supporting the treatment device(s) and associated methods of the present disclosure.

Finally, it will be appreciated that the devices, systems, assemblies, and methods disclosed herein may be delivered endoscopically, transluminally, or percutaneously, as well as used within other access devices such as steerable luminal access devices.

It will be appreciated that all structures, devices, systems, assemblies, and methods discussed herein are examples implemented in accordance with one or more principles of this disclosure, and are not the only way to implement these principles, and thus are not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, assemblies, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. Apparatus facilitating identification, characterization, and/or location of a site treated by application of therapeutic energy thereto, said apparatus comprising
an energy-delivering assembly having an energy-delivering region along which an energy field is generated to apply therapeutic energy to a treatment site; and
a device configured to characterize the treatment site for assessment by a medical professional in determining a treatment protocol generating an energy field with said energy-delivering region.

2. The apparatus of claim 1, wherein said device is configured to deploy a deployable marker at the treatment site.

3. The apparatus of claim 2, wherein the marker is an injectable material delivered through a lumen delivered with said energy-delivering assembly.

4. The apparatus of claim 3, wherein said energy-delivering assembly comprises an energy-delivering member along which said energy-delivering region is defined, and said lumen is independent of said energy-delivering member.

5. The apparatus of claim 4, wherein said lumen is delivered to the treatment site with the energy-delivering member.

6. The apparatus of claim 3, wherein said energy-delivering assembly comprises an energy-delivering member defining the lumen through which the injectable is delivered therethrough.

7. The apparatus of claim 2, wherein the marker is carried by said energy-delivering assembly.

8. The apparatus of claim 7, wherein the marker is delivered to the treatment site as a part of said energy-delivering assembly and separated from said energy-delivering assembly to be deployed at the treatment site.

9. The apparatus of claim 7, wherein the marker is carried over said energy-delivering assembly and slidable off said energy-delivering assembly to be deployed at the treatment site.

10. The apparatus of claim 7, wherein said energy-delivering assembly defines a housing in which the marker is carried to the treatment site and from which the marker is released to be deployed at the treatment site.

11. The apparatus of claim 1, wherein said device profiles a characteristic of the treatment site.

12. The apparatus of claim 11, wherein said device uses one or more of elastography, magnetic resonance electrical impedance tomography, or hyperspectral imaging to map properties of the treatment site.

13. A system for identifying characteristics of a treatment site to aid in performing an energy-based treatment at the treatment site, said system comprising:
an energy-delivering device comprising an energy-delivering assembly with an energy-delivering region along which an energy field is generated to apply therapeutic energy to a treatment site;
a delivery device having a working channel through which said energy-delivering assembly is delivered to a treatment site; and
a device configured to characterize the treatment site for assessment by a medical professional in determining a treatment protocol generating an energy field with said energy-delivering region.

14. The system of claim 13, wherein said device uses one or more of elastography, magnetic resonance electrical impedance tomography, or hyperspectral imaging to map properties of the treatment site, and said system further comprises a processor configured to receive information from said device and to develop models of the treatment site based on the received information.

15. The system of claim 13, wherein said device is configured to deploy a deployable marker at the treatment site.

16. The system of claim 15, wherein said deployable marker is identifiable after an energy field has been applied to the treatment site and the device has been removed from the treatment site.

17. A method for treating and characterizing a treatment site, said method comprising:
delivering an energy-delivering assembly of an energy-delivering device of an energy-delivering treatment system to a treatment site;
generating an energy field along an energy-delivering region of the energy-delivering assembly to apply therapeutic energy to the treatment site;
determining properties or characteristics of the treatment site with the energy-delivering treatment system; and
using the determined properties to affect the treatment being performed, or further treatment to be performed with respect to the treatment site.

18.

19. The method of claim 17, further comprising deploying a marker during or after application of therapeutic energy to the treatment site. wherein determining properties or characteristics comprises determining the location of the treatment site using the marker.

20. The method of claim 17, further comprising using the determined properties to create a three-dimensional map of the properties of the treatment site.

21. The method of claim 19, further comprising using the map of the properties of the treatment site to aid prediction and measurement of therapeutic energy to be applied to a treatment site.
